(19) United States
(12) Patent Application Publication
(10) Pub. No.: US 2019/0127710 A1
(43) Pub. Date: May 2, 2019

(54) TIME RELEASE ENZYMATIC HYDROGEN SULFIDE SCAVENGERS

(71) Applicants: Scott Eric Lehrer, The Woodlands, TX (US); Soma Chakraborty, Houston, TX (US); Prasad Dhulipala, Katy, TX (US)

(72) Inventors: Scott Eric Lehrer, The Woodlands, TX (US); Soma Chakraborty, Houston, TX (US); Prasad Dhulipala, Katy, TX (US)

(73) Assignee: Baker Hughes, a GE company, LLC, Houston, TX (US)

(21) Appl. No.: 15/794,162

(22) Filed: Oct. 26, 2017

(51) Int. Cl. C12N 9/10; C12N 9/02; C10G 31/00; C02F 3/34

(52) U.S. Cl. CPC C12N 9/1085; C12Y 205/01047; C12N 9/0051; C02F 2101/101; C10G 31/00; C02F 3/342; C10G 2300/207; C12Y 108/05004

(57) ABSTRACT

A method of reducing an amount of a sulfur-containing compound in a base fluid comprises contacting the base fluid comprising the sulfur-containing compound with a treatment composite, the treatment composite comprising an enzymatic scavenger which is encapsulated by a encapsulating material, or disposed in a matrix, a container, or a combination thereof; releasing the enzymatic scavenger from the treatment composite; and reducing a number of the sulfur-containing compound in the fluid.

### BACKGROUND

Hydrogen sulfide is a colorless gas with an offensive odor. It is soluble in water and oils. Hydrogen sulfide is often encountered in the oil and gas industry. It can occur naturally as a component of formation gases. Thermal degradation of organic materials and sulfate reducing bacteria (SRB) can also produce hydrogen sulfide. Removal of hydrogen sulfide is warranted because hydrogen sulfide is corrosive, toxic, and flammable.

The process of removing hydrogen sulfide in the oil and gas industry is known as gas sweetening and can be accomplished by either iron sponge H₂S scrubbers or chemical scavengers. Typical hydrogen sulfide scavengers used in the oilfield include amine based scavengers such as triazines, oxidants such as chlorine dioxide, amine-aldehyde condensates, metal carboxylates and chelates.

Despite all the advances, there is a need for alternative hydrogen sulfide scavenger. Since the generation of hydrogen sulfide is often continuous, it would be a further advantage if the alternative hydrogen sulfide can be gradually and consistently released to allow for continuous removal of hydrogen sulfide.

### BRIEF DESCRIPTION

A method of reducing an amount of a sulfur-containing compound in a fluid comprises contacting the base fluid comprising the sulfur-containing compound with a treatment composite, the treatment composite comprising an enzymatic scavenger which is coated by a coating material, or disposed in a matrix, a container, or a combination thereof; releasing the enzymatic scavenger from the treatment composite; and reducing a number of the sulfur-containing compound in the fluid.

A treatment composite comprises an enzymatic scavenger which is coated by a coating material, or disposed in a matrix, a container, or a combination thereof, wherein the enzymatic scavenger comprises a cysteine synthase enzyme, a sulfide quinone reductase enzyme, or a combination comprising at least one of the foregoing; the cysteine synthase enzyme is at least 75% homologous to the cDNA sequence of SEQ ID NO:1, and the sulfide quinone reductase is at least 75% homologous to the cDNA sequence of SEQ ID NO:2.

### BRIEF DESCRIPTION OF THE DRAWINGS

A description of the figures, which are meant to be exemplary and not limiting, is provided in which:

FIG. 1 (SEQ ID NO:1) represents the nucleotide sequence of cysteine Synthase or O-Acetyl Serine Sulfhydrylase (OASS) from Aeropyrum Pernix; and

FIG. 2 (SEQ ID NO:2) represents the nucleotide sequence of a sulfide quinone reductase from Acidithiobacillus ferroxidans.

### DETAILED DESCRIPTION

The inventors hereof have discovered treatment composites that can controllably release enzymatic scavengers. The time release feature allows the scavengers to be applied on a batch basis to a petroleum production operation or wastewater operation but still provides continuous feed of the scavengers.

The treatment composites can be but not necessarily applied downhole. If applied downhole, the treatment composites can be delivered in the form of a solid article such as a solid stick providing an alternative to squeezing the scavengers into a formation.

The use of these treatment composites can also provide an efficiency improvement as compared to traditional hydrogen sulfide scavengers such as triazines. Triazines are typically used in an amount that is greater than ten-fold theoretical values because most of the scavengers are wasted as unreacted scavengers exiting with the treated stream from the wastewater treatment application. The treatment composites as disclosed herein can be applied in a time-release manner so that most of the scavengers are released as wastewater or other fluids are produced resulting in much less loss due to feeding of excess scavengers.

The treatment composites include an enzymatic scavenger which is coated by a coating material, or disposed in a matrix, a container, or a combination thereof. The amount of the enzymatic scavenger in the composite is the amount sufficient to effectuate the desired result over a sustained period of time and may be as low as 1 ppm. Generally, the amount of the enzymatic sulfide scavenger in the composites is from about 1 wt. % to about 90 wt. %, preferably about 30 wt. % to about 90 wt. % or about 35 wt. % to about 70 wt. %, based on the total weight of the treatment composites. The enzymatic scavenger can be present in a solid form. The treatment composites are in the form of particles, pellets, or solid articles such as cylinders, tubes, sticks, and the like. In an embodiment, the treatment composites have a specific gravity of at least about 1.16.

The coating material is dissolvable or degradable in an aqueous based fluid or an oil based fluid that include a sulfur-containing compound to be removed. Thus the enzymatic scavengers can be controllably released. The coating material can be a polymeric material which cured, partially cured, or uncured thermoset or thermoplastic polymers. Exemplary thermoplastics include polyethylene, polypropylene, acrylonitrile-butadiene styrene, polystyrene, polyvinyl chloride, fluoroplastics, polysulfide, styrene acrylonitrile, nylon, and phenylene oxide. Exemplary thermosets include epoxy, phenolic (a true thermosetting resin such as resole or a thermoplastic resin that is rendered thermosetting by a hardening agent), polyester resin, polyurethanes, epoxy-modified phenolic resin, and derivatives thereof.

Exemplary materials for the coating include epoxy, cured cyanate ester, phenolic, melamine-formaldehyde, polyurethane, carbamate, polycarbodiimide, polyamide, polyamide imide, bismaleimide resins, furan resins, polyolefin such as polyethylene, polypropylene, a polyethylene-polypropylene copolycarbonate, polystyrene, or a combination comprising at least one of the foregoing. The phenolic resin includes, e.g., a phenol formaldehyde resin obtained by the reaction of phenol, bisphenol, or derivatives thereof with formaldehyde. The curing agent for the polymer matrix is nitrogen-containing compounds such as amines and their derivatives; oxygen-containing compounds such as carboxylic acid terminated polyesters, anhydrides, phenol-formaldehyde resins, amino-formaldehyde resins, phenol, bisphenol A and cresol novolacs, phenolic-terminated epoxy resins; and catalytic curing agents such as tertiary amines, Lewis acids, Lewis bases; or a combination thereof.

The components of the coating can be present in more than one layer. The compositions for each layer can be the same or different. Optionally, the coating or individual layers are disposed directly on the enzymatic scavengers or other layers, that is, no intervening layers are present other than those described. The coatings and layers can be continuous or discontinuous. To optimize the controlled release of the enzymatic scavengers, the coating covers 80 to 100% of the surface area of the scavengers. For coatings having more than one layer, each layer covers 80 to 100% of the surface area of the scavengers or the underlying layer.

The thickness of the coating is adjusted to provide the desired controlled release of the enzymatic scavengers. In an embodiment, the total thickness of the coating is about 0.1 to about 50 micrometers. Within this range, the thickness may be greater than or equal to about 0.5 micrometer, or greater than or equal to 1 micrometers. Also within this range the thickness may be less than or equal to 25, or less than or equal to 10 micrometers. The amount of the coating is from about 0.5 to about 10% by weight of the coated scavengers.

The coated scavengers can be manufactured by various methods. The scavengers can be coated by spray coating (for example, top, bottom, or side spray coating), drum coating, pan coating, fluid bed coating, continuous pour coating, sputtering, or any other method known to those of skill in the art.

According to an embodiment, the coating is disposed on the enzymatic scavengers by mixing in a vessel, e.g., a reactor. Individual components, e.g., the enzymatic scavengers and polymer materials (e.g., reactive monomers used to form, e.g., an epoxy or polyamide coating) are combined in the vessel to form a reaction mixture and are agitated to mix the components. Further, the reaction mixture is heated at a temperature or at a pressure commensurate with forming the coating.

In another embodiment, the coating is disposed on the particle via spraying such as by contacting the aggregate particles with a spray of the coating material. The coated aggregate particles are heated to induce crosslinking of the coating. Low temperature curing methods may be employed (e.g., using fast setting “cold set” or “cold cure” polymer matrix materials), where heating may be a problem, such as when coating materials and/or enzymatic scavengers may be sensitive to heat. Alternatively, indirect heating processes may be employed with such materials when it is necessary to heat a coating material for cure.

The enzymatic scavengers can also be delivered in a container. The container is made of a degradable or dissolvable polymeric material as described herein for the coating. A weighting agent can be added. The container is designed to protect the enzymatic scavenger until the enzymatic scavenger is substantially in the desired location. The container can also impart time release feature as the container can be degraded or dissolved in a controlled manner thus releasing the enzymatic scavenger over an extended period of time.

The shape of the container is cylindrical, spherical, rectangular, or the like. If the treatment composites are used downhole, any shape that allows passage down a wellbore can be used. The outside diameter of the container should be smaller than the inside diameter of the wellbore. In an embodiment, the outside diameter of the container is about 0.5 inch to about 3 inches or about 1 inch to about 2.5 inches. The container can have any effective thickness, for example from about 0.5 to about 30 millimeters or about 1 to about 20 millimeters or about 5 to about 15 millimeters. For downhole applications, the container must be structurally strong and thus thick enough to resist substantial physical and mechanical forces without breaking. The container may have any length necessary to hold the desired amount of enzymatic scavenger.

In an embodiment, the treatment composite is a tubular body filled with enzymatic scavenger. The tubular body is sealed with a cap, for example, an arcuately shaped, domed closure.

The containers can be made by any conventional methods used in forming polymeric articles. Exemplary methods include thermoforming, molding, casting, extruding, and the like. An open container can be manufactured first. After the open container is filled with enzymatic scavenger, it can be sealed in ways known in the art. Alternatively a closed container is formed first. An aperture is then made, and the enzymatic scavenger is loaded into the container via the aperture. A polymeric plug can be used to seal the container.

The enzymatic scavenger can also be disposed in a matrix. The matrix can be the same degradable or dissolvable material as described herein for the coating. The composites are formed by mixing a quantity of the melted matrix material or a precursor thereof with enzymatic scavenger and other optional components if present. After mixing, the desired weighting agent can be added to the mixture. The mixture can be further processed for example in a mold to obtain a treatment composite having the desired shape. Exemplary shapes include powders, pellets, articles cylindrical, spherical, rectangular, or any other shape that allows passage down a wellbore if the treatment composites are used in a downhole application.

The matrix can also be a porous substrate. In this instance, the enzymatic scavenger is adsorbed or absorbed in the porous substrate. Exemplary porous substrates include a metal oxide, hydrotalcite, nanoclay including bentonite, a zeolite, a molecular sieve, metal organic frameworks (MOF) or a combination comprising at least one of the foregoing. Bentonites used to make the particles include zinc bentonites, calcium bentonites, praseodymium bentonites, or a combination comprising at least one of the foregoing. Zeolites and molecular sieves are commercially available. Exemplary metal oxides include alumina, zirconium oxide and titanium oxide. Alumina is specifically mentioned.

The pore size of the porous substrate is not particularly limited and can vary depending on the particle size of the enzymatic scavenger used and the desired leach rate. The surface area of the porous substrate can be between about 1 m²/g to about 10 m²/g, preferably between from about 1.5 m²/g to about 8 m²/g, the diameter of the porous substrate is between from about 0.1 to about 3 mm, preferably between from about 150 to about 2,000 micrometers, and the pore volume of the porous substrate is between from about 0.01 to about 0.10 cc/g.

The treatment composites can be made by mixing the material having a porous structure and a solution/dispersion of the enzymatic scavenger, and removing the solvent. The enzymatic scavenger powder can also be absorbed or adsorbed into the porous substrate at a pressure that is lower than the atmospheric pressure.

The weight ratio of enzymatic scavenger relative to the matrix is generally between from about 90:10 to about 10:90. When the enzymatic scavenger is disposed in a porous substrate, the composite can contain about 5 wt. % to about 50 wt. %, or about 10 wt. % to about 40 wt. %, or about 20 wt. % to about 40 wt. % of the enzymatic scavenger based on the total weigh of the treatment composite.

The enzymatic scavenger comprises a cysteine synthase enzyme, a sulfide quinone reductase enzyme, or a combination comprising at least one of the foregoing; the cysteine synthase enzyme is at least 75%, at least 80%, at least 90%, or at least 95% homologous to the cDNA sequence of SEQ ID NO:1, and the sulfide quinone reductase is at least 75%, at least 80%, at least 90%, or at least 95% homologous to the cDNA sequence of SEQ ID NO:2. cDNA is defined as DNA synthesized from a messenger RNA (mRNA) template in an enzymatic catalyzed reaction using reverse transcriptase. The enzymatic scavengers are disclosed in U.S. Pat. No. 9,587,159 and U.S. 2016/0039697, the disclosure of both of which is incorporated herein by reference in its entirety.

‘Cysteine synthase enzyme’ is defined herein to be the active site of the cysteine synthase enzyme to convert a sulfur-containing compound such as hydrogen sulfide into L-cysteine and acetate. The active site may be or include the whole protein, an active fragment of the protein, a mimetic of the protein, and combinations thereof. ‘Fragment’ as used herein is meant to include any amino acid sequence shorter than the full-length cysteine synthase enzyme, but where the fragment maintains similar activity to the full-length cysteine synthase enzyme. Fragments may include a single contiguous sequence identical to a portion of the cysteine synthase enzyme sequence. Alternatively, the fragment may have or include several different shorter segments where each segment is identical in amino acid sequence to a different portion of the amino acid sequence of the cysteine synthase enzyme, but linked via amino acids differing in sequence from the cysteine synthase enzyme. ‘Mimetic’ as used herein may include polypeptides, which may be recombinant, and peptidomimetics, as well as small organic molecules, which exhibit similar or enhanced catalytic activity as compared to the cysteine synthase enzyme described herein.

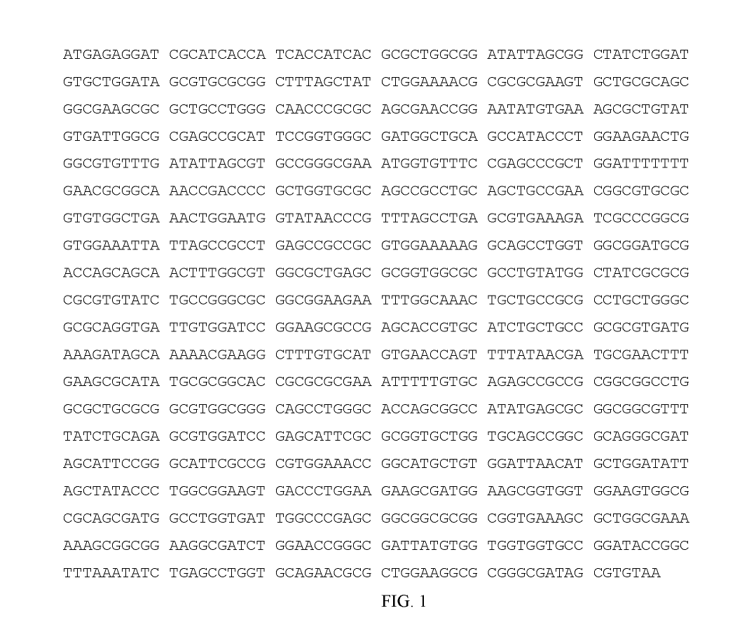

The gene for the cysteine synthase enzyme may be codon optimized to increase the efficiency of its expression in E. coli. The nucleotide sequence of one embodiment of the cysteine synthase enzyme is set forth in FIG. 1 (SEQ ID NO:1). The gene coding for the cysteine synthase enzyme may have a nucleotide sequence that is substantially homologous to the nucleotide sequence of SEQ ID NO:1. The term “substantially homologous” is used herein to denote nucleotides having at least 75% sequence identity to the sequence shown in SEQ ID NO:1, alternatively from about 80% independently to about 99.5%, or from about 85% independently to about 95%.

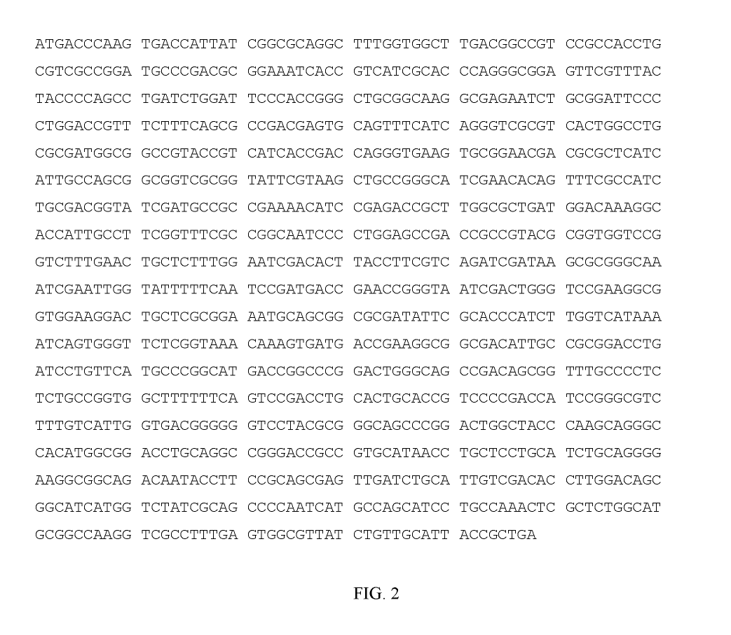

The sulfide quinone reductase (SQR) enzyme used as a enzymatic scavenger in the treatment composite may originate from various organisms. The SQR enzyme prevents the formation of sulfur-containing compounds such as hydrogen sulfide. In a preferred embodiment, the nucleotide sequence encoding the SQR enzyme may be derived from a gram negative, acidophilic and thermophilic bacterium, such as Acidithobacillus ferroxidans, Metallospora cuprina and Metallospora sedula, using polymerase chain reaction (PCR) amplification. A sulfide quinone reductase DNA sequence from Acidithiobacillus ferroxidans is set forth in FIG. 2 (SEQ ID NO:2). The gene coding for the sulfide quinone reductase enzyme may have a nucleotide sequence that is substantially homologous to the nucleotide sequence of SEQ ID NO:2. The term “substantially homologous” is used herein to denote nucleotides having at least 75% sequence identity to the sequence shown in SEQ ID NO:2, alternatively from about 80% independently to about 99.5%, or from about 85% independently to about 95%. The SQR gene sequence was amplified using A. ferroxidans genomic DNA and was cloned in a protein expression vector. A homology may be similar for other SQR enzymes depending on originating organisms.

The treatment composites can further comprise a pyridoxal phosphate, O-acetylserine, dithothreitol, coenzyme Q10, plastoquinone, vitamin K2, or a combination comprising at least one of the foregoing.

When the treatment composites are conveyed downhole in a solid form without using any liquid carrier, weighting agents can be incorporated into the treatment composites. For example, a weighting agent can be included in the container that carries the enzymatic scavenger. A weighting agent can also be dispersed in the matrix that contains the enzymatic scavenger. As used herein weighting agents include barite, rare earth metals such as cerium and lanthanum, hematite, rare earth metal salts such as oxides, hydroxides, carbides. Additional weight agents include calcium carbonate, magnesium carbonate, zinc carbonate, calcium magnesium carbonate, manganese tetra oxide and the like.

The composites containing the enzymatic scavenger can be added to any aqueous or nonaqueous fluids having sulfur-containing compounds sought to be reduced. Such fluids include liquefied petroleum gas, crude oil and petroleum residual fuel, heating oil, a drilling fluid, a servicing fluid, a production fluid, a completion fluid, an rejection fluid, a refinery fluid, wastewater, or a combination comprising at least one of the foregoing. Thus, the treatment composites as disclosed herein are useful in controlling sulfur-containing compounds in water systems, oil and gas production and storage systems, and other similar systems.

As used herein, a sulfur-containing compound include a sulfide such as H₂S, a bisulfide, an organic compound that contains sulfur, or a combination comprising at least one of the foregoing. In an embodiment, the untreated fluid contains greater than about 1 ppm, greater than about 5 ppm, greater than about 10 ppm, greater than about 20 ppm, greater than about 50 ppm, greater than about 100 ppm, or greater than about 200 ppm of the sulfur containing compound.

The treatment composites can be contacted with a fluid including the sulfide-containing compound for removal. Contact can occur in a variety of containers, such as a process or transport line, a separate stirred or non-stirred container or other vessels such as scrubbers or strippers. The composites can also be conveyed downhole to treat various process streams.

Any known method of introducing treatment composites into a well or pipeline can be used. Advantageously, the treatment composites can be simply dropped into the well or pipeline fluid without using any liquid carrier. Because treatment composites have a specific gravity of at least about 1.16, the composites readily sink into the fluid to be treated. The falling velocity may be enhanced by weights, or by altering the size and shape of the treatment composites.

If desired, the treatment composites disclosed herein may also be employed with carrier or treatment fluids in order to facilitate placement of the composite. The carrier fluid may be a brine, sea water, fresh water, a liquid hydrocarbon, or a gas such as nitrogen or carbon dioxide. Suitable carrier fluids include or may be used in combination with fluids have gelling agents, cross-linking agents, gel breakers, surfactants, foaming agents, demulsifiers, buffers, clay stabilizers, acids, or mixtures thereof. The amount of the treatment composites present in a composition containing the composite and carrier fluid is typically between from about 0.5 wt. % to 40 wt. %, about 1 wt. % to about 20 wt. %, or 0.5 to 10 wt. % based on the total weight of the composition.

The treatment composites as disclosed herein may be used in any well treatment operation where the presence of undesirable sulfur-containing compound may be encountered. As such, the well treatment composite may be a component of a fracturing fluid (with or without the presence of a proppant), an acidizing fluid, drilling fluid, completion fluid, acidizing fluid, etc. In addition, the composite may be used during the transport, storage and/or processing of oil or gas to address issues raised by the presence of sulfur-containing compounds.

The treatment composites have time release feature. In particular, the carrier, matrix, and the container in the composites are degradable or dissolvable at a generally constant rate over an extended period of time in water and/or aliphatic and aromatic hydrocarbons. The enzymatic scavengers can also desorb from a porous substrate in a controlled manner into an aqueous or a hydrocarbon fluid. The treatment composites therefore permit a continuous supply of the enzymatic scavenger into the targeted area. Costs of operation are therefore significantly lowered.

Set forth are various embodiments of the disclosure.

### Embodiment 1

A method of reducing an amount of a sulfur-containing compound in a fluid, the method comprising: contacting the fluid comprising the sulfur-containing compound with a treatment composite, the treatment composite comprising an enzymatic scavenger which is coated by a coating material, or disposed in a matrix, a container, or a combination thereof; releasing the enzymatic scavenger from the treatment composite; and reducing a number of the sulfur-containing compound in the fluid.

### Embodiment 2

The method of any of the preceding embodiment, wherein the coating material, the matrix, and the container independently comprise a degradable or dissolvable material comprising an epoxy, a cured cyanate ester, a phenolic, a melamine-formaldehyde, a polyurethane, a carbamate, a polycarbodiimide, a polyamide, a polyamide imide, a bismaleimide, a furan resin, a polyolefin, a polystyrene, or a combination comprising at least one of the foregoing. Alternatively the matrix comprises a porous substrate, and the treatment composite comprises the enzymatic scavenger adsorbed or absorbed in the porous substrate. The porous substrate comprises a metal oxide, hydrotalcite, nanoclay, a zeolite, a molecular sieve, a metal organic framework, or a combination comprising at least one of the foregoing.

### Embodiment 3

The method of any of the preceding embodiment, wherein the treatment composite has a cylindrical or spherical shape.

### Embodiment 4

The method of any of the preceding embodiment, wherein the enzymatic scavenger comprises a cysteine synthase enzyme, a sulfide quinone reductase enzyme, or a combination comprising at least one of the foregoing; the cysteine synthase enzyme is at least 75% homologous to the cDNA sequence of SEQ ID NO:1, and the sulfide quinone reductase is at least 75% homologous to the cDNA sequence of SEQ ID NO:2.

### Embodiment 5

The method of any of the preceding embodiment, wherein the enzymatic scavenger is present in a solid form in the treatment composite.

### Embodiment 6

The method of any of the preceding embodiment, wherein the treatment composite further comprises a pyridoxal phosphate, 0-acetylserine, dithothreitol, coenzyme Q10, plastoquinone, vitamin K2, or a combination comprising at least one of the foregoing.

### Embodiment 7

The method of any of the preceding embodiment, wherein the treatment composite further comprises a weighting agent.

### Embodiment 8

The method of any of the preceding embodiment, wherein the treatment composite comprises about 1 wt. % to about 90 wt. % of the enzymatic scavenger.

### Embodiment 9

The method of any of the preceding embodiment, wherein the fluid is a liquefied petroleum gas, a crude oil, a petroleum residual fuel, a heating oil, a drilling fluid, a servicing fluid, a production fluid, a completion fluid, an rejection fluid, a refinery fluid, wastewater, or a combination comprising at least one of the foregoing.

### Embodiment 10

The method of any of the preceding embodiment, wherein releasing the enzymatic scavenger comprises degrading or dissolving the coating material, the matrix, the container, or a combination thereof. Releasing the enzymatic scavenger can also comprise desorbing the enzymatic scavenger from the porous substrate.

### Embodiment 11

The method of any of the preceding embodiment, wherein the fluid is within a hydrocarbon producing reservoir, and the method further comprises conveying the treatment composite into the reservoir. In an embodiment, the treatment composite is conveyed into the reservoir in a solid form without using a liquid carrier.

### Embodiment 12

A treatment composite comprising an enzymatic scavenger which is coated by a coating material, or disposed in a matrix, a container, or a combination thereof, wherein the enzymatic scavenger comprises a cysteine synthase enzyme, a sulfide quinone reductase enzyme, or a combination comprising at least one of the foregoing; the cysteine synthase enzyme is at least 75% homologous to the cDNA sequence of SEQ ID NO:1, and the sulfide quinone reductase is at least 75% homologous to the cDNA sequence of SEQ ID NO:2.

### Embodiment 13

The treatment composite of any of the preceding embodiment, wherein the treatment composite has a cylindrical or spherical shape.

### Embodiment 14

The treatment composite of claim 16, wherein the coating material, the matrix, and the container independently comprise a degradable or dissolvable material comprising an epoxy, a cured cyanate ester, a phenolic, a melamine-formaldehyde, a polyurethane, a carbamate, a polycarbodiimide, a polyamide, a polyamide imide, a bismaleimide, a furan resin, a polyolefin, a polystyrene, or a combination comprising at least one of the foregoing. Alternatively or in addition, the matrix comprises a porous substrate, and the treatment composite comprises the enzymatic scavenger adsorbed or absorbed in the porous substrate. The porous substrate can comprise a metal oxide, hydrotalcite, nanoclay, a zeolite, a molecular sieve, a metal organic framework, or a combination comprising at least one of the foregoing.

All ranges disclosed herein are inclusive of the endpoints, and the endpoints are independently combinable with each other. As used herein, “combination” is inclusive of blends, mixtures, alloys, reaction products, and the like. All references are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. “Or” means “and/or.” The modifier “about” used in connection with a quantity is inclusive of the stated value and has the meaning dictated by the context (e.g., it includes the degree of error associated with measurement of the particular quantity).

1. A method of reducing an amount of a sulfur-containing compound in a fluid, the method comprising:
contacting the fluid comprising the sulfur-containing compound with a treatment composite, the treatment composite comprising an enzymatic scavenger which is coated by a coating material, or disposed in a matrix, a container, or a combination thereof;
releasing the enzymatic scavenger from the treatment composite; and
reducing a number of the sulfur-containing compound in the fluid.

2. The method of claim 1, wherein the coating material, the matrix, and the container independently comprise a degradable or dissolvable material comprising an epoxy, a cured cyanate ester, a phenolic, a melamine-formaldehyde, a polyurethane, a carbamate, a polycarbodiimide, a polyamide, a polyamide imide, a bismaleimide, a furan resin, a polyolefin, a polystyrene, or a combination comprising at least one of the foregoing.

3. The method of claim 1, wherein the matrix comprises a porous substrate, and the treatment composite comprises the enzymatic scavenger adsorbed or absorbed in the porous substrate.

4. The method of claim 3, wherein the porous substrate comprises a metal oxide, hydrotalcite, nanoclay, a zeolite, a molecular sieve, a metal organic framework, or a combination comprising at least one of the foregoing.

5. The method of claim 1, wherein the treatment composite has a cylindrical or spherical shape.

6. The method of claim 1, wherein the enzymatic scavenger comprises a cysteine synthase enzyme, a sulfide quinone reductase enzyme, or a combination comprising at least one of the foregoing; the cysteine synthase enzyme is at least 75% homologous to the cDNA sequence of SEQ ID NO:1, and the sulfide quinone reductase is at least 75% homologous to the cDNA sequence of SEQ ID NO:2.

7. The method of claim 1, wherein the enzymatic scavenger is present in a solid form in the treatment composite.

8. The method of claim 1, wherein the treatment composite further comprises a pyridoxal phosphate, O-acetylserine, dithothreitol, coenzyme Q10, plastoquinone, vitamin K2, or a combination comprising at least one of the foregoing.

9. The method of claim 1, wherein the treatment composite further comprises a weighting agent.

10. The method of claim 1, wherein the treatment composite comprises about 1 wt. % to about 90 wt. % of the enzymatic scavenger.

11. The method of claim 1, wherein the fluid is a liquefied petroleum gas, a crude oil, a petroleum residual fuel, a heating oil, a drilling fluid, a servicing fluid, a production fluid, a completion fluid, an rejection fluid, a refinery fluid, wastewater, or a combination comprising at least one of the foregoing.

12. The method of claim 1, wherein releasing the enzymatic scavenger comprises degrading or dissolving the coating material, the matrix, the container, or a combination thereof.

13. The method of claim 3, wherein releasing the enzymatic scavenger comprises desorbing the enzymatic scavenger from the porous substrate.

14. The method of claim 1, wherein the fluid is within a hydrocarbon producing reservoir, and the method further comprises conveying the treatment composite into the reservoir.

15. The method of claim 14, wherein the treatment composite is conveyed into the reservoir in a solid form without using a liquid carrier.

16. A treatment composite comprising an enzymatic scavenger which is coated by a coating material, or disposed in a matrix, a container, or a combination thereof, wherein the enzymatic scavenger comprises a cysteine synthase enzyme, a sulfide quinone reductase enzyme, or a combination comprising at least one of the foregoing; the cysteine synthase enzyme is at least 75% homologous to the cDNA sequence of SEQ ID NO:1, and the sulfide quinone reductase is at least 75% homologous to the cDNA sequence of SEQ ID NO:2.

17. The treatment composite of claim 16, wherein the treatment composite has a cylindrical or spherical shape.

18. The treatment composite of claim 16, wherein the coating material, the matrix, and the container independently comprise a degradable or dissolvable material comprising an epoxy, a cured cyanate ester, a phenolic, a melamine-formaldehyde, a polyurethane, a carbamate, a polycarbodiimide, a polyamide, a polyamide imide, a bismaleimide, a furan resin, a polyolefin, a polystyrene, or a combination comprising at least one of the foregoing.

19. The treatment composite of claim 16, wherein the matrix comprises a porous substrate, and the treatment composite comprises the enzymatic scavenger adsorbed or absorbed in the porous substrate.

20. The treatment composite of claim 19, wherein the porous substrate comprises a metal oxide, hydrotalcite, nanoclay, a zeolite, a molecular sieve, a metal organic framework, or a combination comprising at least one of the foregoing.


SEQUENCE LISTING (2 sequences)

SEQ ID NO: 1 - DNA, 1197 bp, Aeropyrum pernix
atgagaggatcgcatcaccatcaccatcacgcgctggcggatattagcggctatctggatgtgctggatagcgtgcgcggctttagctatctggaaaacgcgcgcgaagtgctgcgcagcggcgaagcgcgctgcctgggcaacccgcgcagcgaaccggaatatgtgaaagcgctgtatgtgattggcgcgagccgcattccggtgggcgatggctgcagccataccctggaagaactgggcgtgtttgatattagcgtgccgggcgaaatggtgtttccgagcccgctggatttttttgaacgcggcaaaccgaccccgctggtgcgcagccgcctgcagctgccgaacggcgtgcgcgtgtggctgaaactggaatggtataacccgtttagcctgagcgtgaaagatcgcccggcggtggaaattattagccgcctgagccgccgcgtggaaaaaggcagcctggtggcggatgcgaccagcagcaactttggcgtggcgctgagcgcggtggcgcgcctgtatggctatcgcgcgcgcgtgtatctgccgggcgcggcggaagaatttggcaaactgctgccgcgcctgctgggcgcgcaggtgattgtggatccggaagcgccgagcaccgtgcatctgctgccgcgcgtgatgaaagatagcaaaaacgaaggctttgtgcatgtgaaccagttttataacgatgcgaactttgaagcgcatatgcgcggcaccgcgcgcgaaatttttgtgcagagccgccgcggcggcctggcgctgcgcggcgtggcgggcagcctgggcaccagcggccatatgagcgcggcggcgttttatctgcagagcgtggatccgagcattcgcgcggtgctggtgcagccggcgcagggcgatagcattccgggcattcgccgcgtggaaaccggcatgctgtggattaacatgctggatattagctataccctggcggaagtgaccctggaagaagcgatggaagcggtggtggaagtggcgcgcagcgatggcctggtgattggcccgagcggcggcgcggcggtgaaagcgctggcgaaaaaagcggcggaaggcgatctggaaccgggcgattatgtggtggtggtgccggataccggctttaaatatctgagcctggtgcagaacgcgctggaaggcgcgggcgatagcgtgtaa

SEQ ID NO: 2 - DNA, 1128 bp, Acidithiobacillus ferrooxidans
atgacccaagtgaccattatcggcgcaggctttggtggcttgacggccgtccgccacctgcgtcgccggatgcccgacgcggaaatcaccgtcatcgcacccagggcggagttcgtttactaccccagcctgatctggattcccaccgggctgcggcaaggcgagaatctgcggattcccctggaccgtttctttcagcgccgacgagtgcagtttcatcagggtcgcgtcactggcctgcgcgatggcggccgtaccgtcatcaccgaccagggtgaagtgcggaacgacgcgctcatcattgccagcggcggtcgcggtattcgtaagctgccgggcatcgaacacagtttcgccatctgcgacggtatcgatgccgccgaaaacatccgagaccgcttggcgctgatggacaaaggcaccattgccttcggtttcgccggcaatcccctggagccgaccgccgtacgcggtggtccggtctttgaactgctctttggaatcgacacttaccttcgtcagatcgataagcgcgggcaaatcgaattggtatttttcaatccgatgaccgaaccgggtaatcgactgggtccgaaggcggtggaaggactgctcgcggaaatgcagcggcgcgatattcgcacccatcttggtcataaaatcagtgggttctcggtaaacaaagtgatgaccgaaggcggcgacattgccgcggacctgatcctgttcatgcccggcatgaccggcccggactgggcagccgacagcggtttgcccctctctgccggtggcttttttcagtccgacctgcactgcaccgtccccgaccatccgggcgtctttgtcattggtgacggggggtcctacgcgggcagcccggactggctacccaagcagggccacatggcggacctgcaggccgggaccgccgtgcataacctgctcctgcatctgcaggggaaggcggcagacaataccttccgcagcgagttgatctgcattgtcgacaccttggacagcggcatcatggtctatcgcagccccaatcatgccagcatcctgccaaactcgctctggcatgcggccaaggtcgcctttgagtggcgttatctgttgcattaccgctga